(19) United States
(12) Patent Application Publication
(10) Pub. No.: US 2004/0005359 A1
(43) Pub. Date: Jan. 8, 2004

(54) Controlled release oral dosage form

(76) Inventors: Xiu Xiu Cheng, Davie, FL (US); Chih Ming Chen, Davie, FL (US); Dacheng Tian, Miramar, FL (US)

Correspondence Address: Martin P. Endres, Esq., HEDMAN & COSTIGAN, P.C., 1185 Avenue of the Americas, New York, NY, 10036, US

(21) Appl. No.: 10/183,776

(22) Filed: Jun. 27, 2002

(51) Int. Cl. A61K 31/554; A61K 31/277; A61K 31/455; A61K 9/24

(52) U.S. Cl. 424/471; 514/211.07; 514/355; 514/523

(57) ABSTRACT

A sustained-release pharmaceutical preparation is disclosed in which a calcium channel blocker, preferably verapamil, core is surrounded by an optional seal coat layer and a water-insoluble coating.

### BACKGROUND OF THE INVENTION

1. Field of the Invention

The present invention relates to oral controlled release dosage formulations containing a calcium channel blocking agent. More specifically, the present invention relates to an oral dosage formulation in the form of a coated single composition core tablet comprising a calcium channel blocking agent, such as amlodipine, diltiazem, nicardipine, nifedipine, verapamil, felodipine, isradipine, nisoldipine, nimodipine, nilvadipine, flunarizine, norverapamil, nitredipine, cinnarizine, fendiline or their pharmaceutically acceptable derivatives, salts and stereoisomers. Preferably, the calcium channel blocking agent is verapamil.

2. Description of the Prior Art

Verapamil (+/−)-5-[(3,4-Dimethoxyphenethyl)methylamino]-2-(3,4-dimethoxyphenyl)-2-isopropylvaleronitrile monohydrochloride or iproveratril is a nondihydropyridine calcium channel blocking agent that inhibits the transmembrane influx of extracellular calcium ions across the membranes of the myocardial cells and vascular smooth muscle cells, without changing serum calcium concentrations. By inhibiting calcium influx, verapamil inhibits the contractile processes of cardiac and vascular smooth muscle, thereby dilating the main coronary and systemic arteries. Verapamil is a class IV antiarrhythmic. It reduces afterload and myocardial contractility.

Verapamil is used orally to treat Prinzmetal variant angina and unstable and chronic stable angina pectoris, the management of hypertension, for the prevention of recurrent premature supraventricular tachycardia (PSVT) and, in combination with a cardiac glycoside, to control ventricular rate at rest and during stress in patients with atrial flutter and/or fibrillation. It has been used as adjunctive therapy in the management of hypertrophic cardiomyopathy or in certain patients after myocardial infarction when beta-adrenergic blocking agents are ineffective or contraindicated for the relief of ongoing ischemia. Benefit has also been demonstrated in the management of manic manifestations of bipolar disorder.

Numerous techniques are in the prior art for preparing sustained or controlled release pharmaceutical formulations. One common technique involves surrounding an osmotically active drug core with a semipermeable membrane or wall. The drug is released from the core by allowing a fluid such as gastric or intestinal fluid to permeate the coating membrane and dissolve the drug so dissolved drug can permeate the membrane. In some cases a hydrogel is employed to push the active ingredient through the passageway in the membrane. The hydrogel imbibes fluid and expands thereby pushing the active ingredient through the membrane or passageway formed in the membrane and from the dosage form.

Several different sustained release formulations of calcium channel blocking agents are patented. Commercially available extended-release tablets of verapamil hydrochloride (Covera-HS®) contain drug in an osmotic delivery system consisting of an osmotically active core surrounded by a semipermeable membrane with a laser-drilled delivery orifice. The core itself is divided into 2 layers: an active drug layer and a push layer containing pharmacologically inert (but osmotically active) components. However, the manufacturing of this multi-layer core system proves to be difficult and expensive. This system, disclosed in U.S. Pat. No. 5,252,338 is incorporated herein by reference. Variations on the osmotic system are also disclosed in U.S. Pat. No. 4,783,337 and 4,753,802 which are incorporated herein by reference.

Another sustained release formulation disclosed in U.S. Pat. No. 4,863,742 (incorporated herein by reference) requires a core comprised of layers of polymeric material superimposed on layers of verapamil. The core is surrounded by a multi-layer membrane with a major portion of a water soluble polymer and a minor portion of a water insoluble polymer. The formulation contains a first component that provides an effective amount of verapamil within one hour after administration and a second component that provides an effective amount of verapamil 6-16 hours after administration.

One limitation associated with these prior art dosage forms is that many of the multi-walled preparations described above do not provide a therapeutic release of the drug prior to initiation of sustained release, which is important when biphasic release profiles are desired.

Other systems are essentially “delayed” release mechanisms wherein there is a delay of drug release in the stomach but once the coated drug reaches the intestines, the release of medication is rapid.

Verapamil is also available as controlled and extended release capsules (Verelan SR®) containing pellets and as extended release caplets (Calan SR®, Isoptin SR®). The extended release capsules contain controlled release beads, a portion of which are uncoated for immediate release. The remainder consists of a drug core with rate-controlling polymeric coating. The extended release caplets contain alginate which swells in water and forms a gelatinous like substance. Verapamil is released both by diffusion out of the gel and by erosion of the tablet. Verapamil shows linear pharmacokinetics for single dosing, but saturation and increased bioavailability with multiple dosing. Because of their pharmacokinetic profile, these are dosed early in the morning, rather than at bedtime. For many patients, these are dosed every twelve hours in the morning and in the evening. Additionally, the caplets should be administered with food to prevent wide differences between peak and trough serum verapamil concentrations.

The need exists for a sustained release pharmaceutical preparation which provides constant blood levels, and is simply and economically produced. Such a delivery dosage form has a practical application, and it represents a valuable contribution to the medical arts. The present invention provides such a composition, and offers an efficient and cost effective method of preparation.

Accordingly, it is an object of this invention to provide a novel and useful dosage form for administering a calcium channel blocker, preferably verapamil, that represents an unexpected improvement in the art and substantially overcomes the disadvantages known to the prior art.

It is an object of this invention to provide a sustained release form of a calcium channel blocker, preferably verapamil, suitable for bedtime administration, which is substantially invariable from subject to subject, and provides significant plasma levels of the calcium channel blocker which are maintained for an extended period after administration.

Another object of the invention is to provide bedtime dosing of verapamil resulting in therapeutic morning levels to reduce the early morning rise in blood pressure and heart rate.

Another object of this invention is to provide 24 hour control of hypertension and angina.

Other objects, features and advantages of the invention are not taught in the prior art but will be more apparent to those versed in the art from the following specification, taken in conjunction with the drawings and the accompanying claims.

### BRIEF SUMMARY OF THE INVENTION

The present invention provides a controlled release calcium channel blocker tablet for oral administration comprising a single composition core tablet coated with a water insoluble film. The core of the tablet comprises a calcium-channel blocking agent and a polymer. The core is optionally seal coated with a water soluble or water dispersible film and then coated with a water insoluble film. There is no separate and distinct push composition which pushes the calcium channel blocker from the dosage form. Instead, the core comprises a substantially homogeneous mixture of calcium channel blocker, polymers, preferably osmopolymers, which will interact with water and aqueous biological fluids causing the polymer to expand or swell, and osmagents (water-soluble osmotic agents which exhibit an osmotic pressure gradient across the water insoluble semipermeable wall of the osmotic device). The subject invention may also include a binder, a lubricant and at least one organic acid which can help buffer the microenvironment of a tablet in solution, either in vitro or in vivo. By using a buffer to regulate the pH, the rate of release can be more precisely controlled. The water-insoluble film coating is preferably a semipermeable film comprising a water insoluble polymer such as cellulose acetate.

Pharmaceutical compositions of the present invention may preferably comprise combinations of low and high molecular weight polymers selected from the group consisting of polyethylene oxides (PEO). Low molecular weight polyethylene oxides, generally defined as those with molecular weights less than 0.5×10⁶, can provide a constant release rate for the active drug by means of forming a gel. High molecular weight PEOs, defined as those with molecular weights of 4×10⁶ and higher, provide delayed drug release by a controlled diffusion. The diffusion is linearly dependent on the molecular weight of the PEO; the higher the molecular weight, the smaller the amount of drug released.

In a preferred embodiment, the core comprises about 70 to 98 weight percent of the total dosage form and preferably about 85 to 96 weight percent of the dosage form. The calcium channel blocker comprises about 30 to 50 weight percent of the total dosage form and preferably 35 to 45 weight percent of the dosage form.

Optimally, this formulation provides 24 hour efficacy with once-daily dosing, with at least 50% of the peak antihypertensive effect remaining at the end of the dosing interval. Release of the calcium channel blocker does not occur until about two hours after administration. The product peaks and maintains levels from 10-24 hours. The usual dosage range is 80 mg-480 mg.

### BRIEF DESCRIPTION OF THE DRAWINGS

FIG. 1 is a graph depicting the dissolution profile in simulated intestinal fluid (pH 6.8) and simulated gastric fluid (pH 1.2) of the formulation as described in Example 1 as tested according to the procedure described in United States Pharmacopoeia (USP) XXIII, Apparatus 2 @ 75 rpm.

FIG. 2 is a graph depicting the dissolution profile in a pH 7.5 buffer of the formulation in Example 2 as tested according to the procedure described in USP XXIII, Apparatus 2 @ 75 rpm.

FIG. 3 is a graph depicting the dissolution profile in a pH 7.5 buffer of the formulation as described in Example 2 as tested according to the procedure described in USP XXII, Apparatus 2 @ 75 rpm.

FIG. 4 is a graph depicting the linear plot of the mean plasma verapamil concentration vs. time of the formulation described in Example 1 and the linear plot of the mean plasma verapamil concentration versus time of the commercially available form of verapamil under fasting conditions.

FIG. 5 is a graph depicting the linear plot of the mean plasma verapamil concentration vs. time of the formulation described in Example 1 and the linear plot of the mean plasma verapamil concentration versus time of the commercially available form of verapamil under fed conditions.

### DETAILED DESCRIPTION OF THE PREFERRED EMBODIMENTS

The subject invention concerns a formulation and dosage form for providing controlled-release of a calcium channel blocker as a active pharmaceutical ingredient. The dosage form is preferably a tablet which comprises a substantially homogeneous core having an active pharmaceutical ingredient, for example a calcium channel blocker, preferably verapamil, homogeneously admixed with a polymer, preferably an osmopolymer which swells when contacted by an aqueous medium. The core can be coated with an insoluble coating and can optionally include a seal coat dispersed between the core and the water soluble coating.

A preferred embodiment of the subject invention provides a controlled release calcium channel blocker formulation for oral administration comprises:

(i) a core comprising

(a) a calcium channel blocker or pharmaceutically acceptable salt thereof

(b) an organic acid,

(c) an osmopolymer,

(d) a binder,

(e) an osmagent, and

(f) a lubricant,

(ii) optionally a seal surrounding said core, and

(iii) a water-insoluble coating comprising:

(a) a water insoluble polymer,

(b) a plasticizer, and

(c) a dissolution enhancing agent

said formulation providing controlled release over a 24 hour period following oral administration.

The core comprises an active pharmaceutical ingredient, for example a calcium channel blocker, and a polymer, for example an osmopolymer. The calcium channel blocker can be selected from the group consisting of verapamil hydrochloride, diltiazem, amlodipine, nicardipine, nifedipine, nisoldipine, niimodipine, nilvadipine, flunarizine, norverapamil, isradipine, feldopine, nitredipine, cinnarizine, fendiline or their pharmaceutically acceptable derivatives, salts and stereoisomers. Most preferably, the calcium channel blocker is verapamil.

Osmopolymers are swellable, hydrophilic polymers which interact with water and aqueous biological fluids causing the osmopolymer to swell or expand and retain water in the polymer structure. Preferably, the osmopolymers in the core are selected from the group consisting of polyethylene oxides. The preferred polyethylene oxides are those with higher molecular weight (MW) grades (4×10⁶ and higher) that provide delayed drug release via the hydrophilic matrix. The drug release proceeds as a controlled diffusion, linearly dependent on the molecular weight of the polyethylene oxide (PEO); the higher the MW, the smaller the amount of drug released. The preferred PEO is Poly-ox WSR Coagulant (MW 5,000,000 viscosity 5,500-7,500 (mPas).

The core can further include an organic acid. Weak organic acids can provide a buffer effect and create a stable pH microenvironment when the pharmaceutical dosage form is contacted by aqueous media. The control of the pH is an important feature which creates a pharmaceutical dosage form which exhibits uniformity and complete drug release with over 80% of the verapamil released within twelve hours. The organic acids are preferably selected from the group consisting of adipic acid, ascorbic acid, succinic acid, citric acid monohydrate, malic acid and tartaric acid, fumaric acid. The preferred organic acid is fumaric acid. Generally, the ratio (wt./wt.) of active ingredient to organic acid will be in the range from 10:1 to 1:1, with from 9:1 to 6:1 being preferred.

The core can also comprise a binder. The binder may be chosen from those materials commonly known in the art. Suitable binders can be polyvinylpyrrolidone (PVP), alginates, methylcellulose, hyroxypropyl methylcellulose, starch and zein. Polyvinylpyrrolidone (PVP K-30) (povidone) (average molecular weight 50,000) is the preferred binder.

Preferred water soluble resins are those with molecular weights less than 0.6×10⁶. Most preferred is Poly-ox WSR N-80 (MW 200,000; viscosity 65-115 at 25° C. (mPas)). When the drug is combined with the higher molecular weight polyethylene oxides and low molecular weight polyethylene oxides, the release is controlled by the swelling of the polymer as well as their erosion, thereby ensuring a constant rate of delivery over a 24 hour period. An optimal concentration of the high molecular weight to low molecular weight polyethylene oxides was determined to be a mixture of almost 1:1.24 (wt./wt.).

The core can also include a water-soluble osmotic agent or osmagent which can be osmotically effective solute, osmotically effective compounds, or other osmotic agents. Osmagents control the release rate of drug by regulating the swelling of osmopolymers. Suitable osmagents for this invention include various organic compounds and inorganic salts, such as magnesium sulfate, magnesium chloride, lithium chloride, potassium sulfate, sodium chloride, sodium carbonate, sodium sulfite, lithium sulfite, potassium chloride, calcium bicarbonate, sodium sulfate, calcium sulfate, potassium acid phosphate, calcium lactate sucrose, lactose dextrose, mannitol, fructose and dextrose. A preferred osmagent is sodium chloride.

Tablet lubricants that can be present in the core are preferably selected from the group consisting of glyceryl monostearates, magnesium stearate, calcium stearate or stearic acid. Preferably, magnesium stearate is present as a lubricant to prevent the powder from agglomerating during processing. In the preferred embodiment, magnesium stearate can be used in an amount of less than 1% of the total core weight.

In a preferred embodiment of the invention, the core of the present invention comprises the following ingredients and percent ranges:

| INGREDIENT | PREFERRED | MOST PREFERRED |
|---|---|---|
| Calcium channel | 30-50% | 35-45% |
| blocker | | |
| Organic acid | 2-10% | 4-8% |
| Low MW | 10-35% | 15-25% |
| osmopolymer | | |
| High MW | 10-20% | 13-18% |
| osmopolymer | | |
| Binder | 2-10% | 3-6% |
| Osmagent | 2-10% | 3-8% |
| Lubricant | 0.1-2% | 0.5-1.5% |

The water-insoluble coating in the preferred embodiment is formed from a water-insoluble polymer, preferably a cellulose polymer selected from the group consisting of a cellulose ether, cellulose ester, or a cellulose ester-ether. Representative materials include a member selected from the group consisting of cellulose acylate, cellulose deacylate, cellulose triacylate, cellulose acetate, cellulose diacetate, cellulose triacetate, mono, di and tricellulose alkanylates, mono, di and tri cellulose aroyllates, and the like. Here, the preferred embodiment comprises cellulose acetate (CA) (398-10) having a degree of substitution up to 1 and an acetyl content of 39.8%, 38% viscosity and a molecular weight of 40,000 (polystyrene equivalents).

The water-insoluble coating can include a plasticizers which affects the water absorption behavior and adhesive property of the film coating and, thereby, the release profile. The plasticizer used in the water-insoluble coating may also function as a flux enhancer that aids in governing fluid flux. Plasticizers which may be used include polyalkylene oxides, such as polyethylene glycols, polypropylene glycols, polyethylene-propylene glycols, and organic plasticizers with low molecular weights, such as glycerol, glycerol monoacetate, diacetate or triacetate. Glycols such as polyethylene glycol assist dispersion and are preferred. Most preferred is polyethylene glycol. In the preferred embodiment, the flux enhancer polyethylene glycol comprises a molecular weight range of 150 to 7500.

Organic esters of plasticizers may also be included in the water-insoluble coating to affect water absorption, adhesive property and plasticizer permanence. Some preferred plasticizers are from the group of organic esters such as triacetin, diethyl phthalate, dibutyl phthalate, triethyl citrate , acetyl tributyl citrate and tributyl citrate. In the preferred embodiment a 1:1 ratio of plasticizer to organic ester of plasticizer found to optimize plasticizer performance and maximize plasticizer permanence, is most preferred. The adhesion (tack value) of the coating is optimal when the concentration of the coating is about 10-20%.

The water-insoluble coating may also contain a dissolution enhancing agent. Some dissolution enhancing agents are polyethylene glycol, sucrose, lactose, fructose or sorbitol. The preferred dissolution-enhancing agents are micronized sucrose or polyethylene glycol. The release rate of verapamil increases as the particle size of the sugar and the amount of plasticizer decreases. In the preferred embodiment, the optimal amount of sucrose is found to be about 5-10%. Six times micronized confectioner's sugar can be added to the coating since the micronized ingredients of similar particle size must be blended in the wet granulation.

Where a color is employed, the color will be applied together with the water-insoluble coating. Examples of coloring agents include known azo dyes, organic or inorganic pigments, or coloring agents of natural origin. Inorganic pigments are preferred, such as the oxides of iron or titanium. The most preferred coating colorant consists of Opadry white YS-1-7003.

The optional seal coating for the core influences the tablet moisture, surface roughness, and coating efficacy and uniformity. The seal coating formulation preferably is an aqueous film coating of polymers, plasticizers and pigments, which may be 1-5% of the formulation. The preferred seal coating formulation on the verapamil core tablet comprises hydroxypropyl methylcellulose. The seal coating can be applied for example, as hudroxypropyl methylcellulose (HPMC) or coating which comprise HPMC, for example compositions sold under the trade name Opadry® (Colorcon, West Point Pa.). A preferred coating is Opadry White® (YS-1-7003. The suspension contains 8% solids wt./wt. In the preferred embodiment, the seal coating constitutes approximately 3-4% of the tablet formulation and has a thickness of 10-15 μm.

The pharmaceutical preparations of the invention can be prepared by compounding the above mentioned components at levels described above, respectively, and using formulation techniques that are well known in the art. In a preferred embodiment, the verapamil or pharmaceutically acceptable salt thereof, organic acid, sodium chloride, Polyox WSR N-80 and Polyox WSR-Coagulant are blended to form a substantially homogenous mixture. The blend is then charged into a top-spray fluidized bed to wet granulate using a binding solution. The binding solution prepared by dissolving povidone K-30 in purified water is sprayed onto the material in a fluid bed granulator. The granules are then dried in the fluidized bed and passed through an oscillator equipped with a screen using a milling machine. Magnesium stearate is then added to the granules before tableting. The core tablets were made using a Manesty Betapress (Liverpool, UK), with a 7/16″ round, standard concave punches. The core tablets were then seal coated with an Opadry White aqueous solution in a perforated pan coater. Finally the seal coated tablets were coated with the water insoluble coating comprising a wall forming suspension in a Glatt GPCG-5 fluidized bed coater. The wall forming suspension comprises cellulose acetate, triacetin, polyethylene glycol 400 and sucrose (confectioner's 6×-micronized) dissolved and suspended in acetone. The cellulose acetate must be added to the solvent. The drug is released slowly and steadily from the hydratable diffusion barrier for a prolonged period of up to 24 hours. The rate of verapamil release was measured in vitro as a dissolution rate according to the USP XXIV for controlled release tablets. The amount of released drug was determined spectrophotometrically at λ-280 nm. The dissolution conditions were USP Apparatus 2, 75 rpm with 900 ml of simulated gastric fluid (0.1 N hydrochloric acid solution at pH 1.2). Dissolution samples were collected at 0, 1, 2, 3, 4, 6, 8, 10, 12, 14 and 16 hours. The controlled release dosage forms prepared according to the present invention should exhibit the following dissolution profile when tested in a USP type 2 apparatus at 75 rpm in simulated gastric fluid (pH 1.22) and at 37° C.:

| Time (hours) | Preferred | Most Preferred |
|---|---|---|
| 2 | 0-25% | 5-20% |
| 4 | 10-40% | 15-35% |
| 8 | 40-75% | 50-65% |
| 12 | *NLT 50% | NLT 60% |
| 14 | NLT 60% | NLT 70% |
| 16 | NLT 65% | NLT 65% |

*NLT = Not Less Than

Using the aforementioned dissolution conditions, the controlled release dosage form prepared according to the present invention exhibits the following dissolution profile when tested in a USP type 2 apparatus at 75 rpm in 900 ml of simulated intestinal fluid (pH 6.8 phosphate buffer) and at 37° C.:

| Time (hours) | Preferred | Most Preferred |
|---|---|---|
| 2 | 0-25% | 5-20% |
| 4 | 10-50% | 15-40% |
| 8 | 30-80% | 40-70% |
| 12 | *NLT 50% | NLT 55% |
| 16 | NLT 60% | NLT 65% |

*NLT = Not Less Than

The invention provides a method of controlling or treating hypertension and angina to provide a maximum blood pressure lowering effect within 6 to 16 hours following administration. With bedtime dosing the invention provides extra blood pressure reduction in the morning and provides 24 hour control of hypertension and angina.

The following examples illustrate the present invention and are not intended to limit the scope of the present invention.

### EXAMPLE 1

A 5.5 kg batch of the formulation was manufactured using all materials which comply with current USP/NF compendial specifications.

A controlled release oral verapamil dosage form in accordance with the present invention is prepared by forming an active core having the following composition:

I ACTIVE CORE

| | |
|---|---|
| Verapamil HCL | 41.59% |
| Polyox N-80 | 20.86% |
| PVP k30 | 4.55% |
| Polyox coagulant | 16.77% |
| Sodium chloride | 7.29% |
| Fumaric acid | 8.32% |
| Magnesium stearate | 0.62% |

The active core is prepared by mixing 2.9 kg of verapamil hydrochloride, USP, 0.508 kg of sodium chloride, USP, and 0.580 kg of fumaric acid, NF in a blender or suitable mixing apparatus for fifteen minutes. This is added to a fluidized bed (Glatt GPCG) processor. Add 0.317 kg of povidone, USP (Kollidon® 30) to 8.740 kg of purified water until it completely dissolves. This solution serves as the granulation fluid and is sprayed onto the verapamil, sodium chloride, fumaric acid mixture in the fluid bed processor until a moist granular mass is obtained. This is then dried in the fluidized bed until the loss on drying is less than 2% and passed through an oscillator equipped with a 20 mesh screen. Pass 1.147 kg of Polyox WSR N-80, NF and 0.922 kg of Polyox WSR Coagulant, NF through a 20 mesh screen. Place into a blender according to the following order and blend for 15 minutes: the verapamil granules as prepared above, Polyox WSR N-80 and Polyox WSR coagulant, NF. Pass 0.034 kg of magnesium stearate through a #40 mesh screen and add the magnesium stearate to the blender. Blend for five minutes at 32 rpm. Compress the blend into tablets on a suitable tableting machine using round concave tooling of 7/16″ in diameter.

II SEAL COATING

| | |
|---|---|
| Opadry white (YS-1-7003) | 100% |

Add 0.090 kg of Opadry white into 1.035 kg of purified water and stir until a homogenous dispersion is obtained. Load the core verapamil tablets into a perforated pan coater and spray the aqueous coating suspension onto the tablets until a 3-5% coating level is achieved.

(NB: Need the mg amount of this coating to figure out the CA coating- the amounts below are off by this amount- together the seal and CA coat comprise 25.12% of the total tablet weight).

III SR COATING

| | |
|---|---|
| Cellulose acetate | 80% |
| PEG 400 | 5% |
| Sucrose | 10% |
| Triacetin | 5% |

The seal coated active tablets were coated with a wall forming suspension in a Glatt GPCG-5 fluidized bed coater. The wall forming suspension comprises cellulose acetate, triacetin, polyethylene glycol 400 and confectioner's sugar.

Disperse cellulose acetate (398-10) in a quantity equivalent to 121.8 mg/tablet or 20% of the total tablet weight into acetone, NF while homogenizing until a clear solution is formed. Add the PEG 400 in a quantity equivalent to 7.61 mg/tablet or 1.25% of the total tablet weight into the solution and mix with a homogenizer for 2 minutes. Add triacetin, USP in an amount equivalent to 1.25% of the total tablet weight, and mix with a homogenizer for 2 minutes. Add confectioner's sugar (six times micronized) in an amount equivalent to 2.5% of the total tablet weight into the solution while stirring for at least ten minutes until it is well dispersed. The inlet temperature is set at 55+/−5° C., the spray rate is 10-20 ml/min, the pan speed is 5-8 rpm with 1 spray gun; the atomization pressure is 37+/−5 psi; the exhaust temperature is 40° C.

Load the seal coated verapamil tablets into a Glatt fluidized bed coater and spray the coating suspension onto the tablets under the following conditions: pump rate of 50-150 ml/min, air velocity of 600-800 M³/h, atomization pressure of 3 bar; inlet temperature of 16-24° C. The coating is continued until a theoretical coating of approximately 3-4% is obtained. After all the coating suspension is consumed and the theoretical coating level is obtained, dry the tablets.

The resulting controlled release tablets were tested in a simulated gastric fluid according to the procedures reported in USP XXIII, Apparatus 2, 37° C. @ 75 rpm and found to have the following release profile:

| Time (hours) | % Released |
|---|---|
| 1 | 1 |
| 2 | 5 |
| 3 | 14 |
| 4 | 24 |
| 6 | 42 |
| 8 | 58 |
| 10 | 69 |
| 12 | 76 |
| 14 | 81 |
| 16 | 84 |

The resulting controlled release tablets were tested in simulated intestinal fluid (pH 6.8 phosphate buffer) according to the procedure described in USP XXIII, using Apparatus 2, 37° C. at 75 rpm and found to have the following release profile:

| Time (hours) | % Released |
|---|---|
| 1 | 1 |
| 2 | 9 |
| 3 | 20 |
| 4 | 30 |
| 6 | 48 |
| 8 | 61 |
| 10 | 71 |
| 12 | 76 |
| 14 | 79 |
| 16 | 82 |

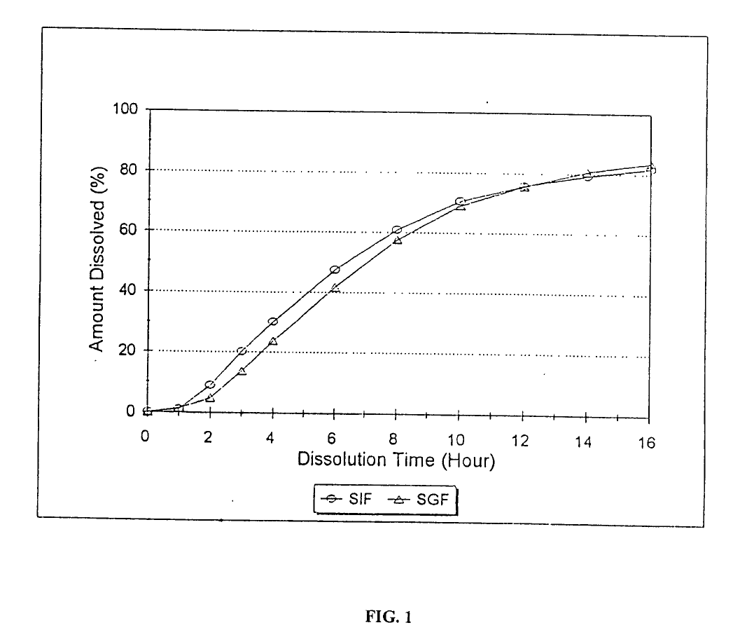

The release profile of the controlled release product shown in this Example is shown in FIG. 1.

Verapamil tablets, such as those produced in Example 1, were analyzed in a six patient test using standard techniques known to the art. Verapamil was first detected in the plasma more than 2 hours after administration, and showed sustained release over 24 hours.

The bioavailability of the controlled release verapamil formulation prepared in this example has been determined under both fed and fasting conditions. Two panels of six patients each were randomly assigned to receive either the verapamil formulation described herein or COVERA® HS in a open, randomized single dose study. Blood samples were collected over a 36 hour period and analyzed for verapamil concentrations with a validated HPLC method.

For the blood level studies carried out Cₘₐₓ is the maximum blood level concentration of the verapamil, Tₘₐₓ is the time at which the maximum blood level concentration occurs and AUC is the “area under the curve” of time versus blood concentration. The results provided are given in Tables 1 and 2 show that the dissolution of a 240 mg single dose of the controlled release verapamil formulation prepared in Example 1 and COVERA® HS is similar under both fed and fasting conditions. Under fasting conditions, the mean Cₘₐₓ and area under the curve (AUC₀₋ₜ) for the Example 1 formulation were 64.4 μg/L and 727.72 μg/L, and for the COVERA® HS were found to be 58.8 μg/L and 730.28 μg/L, respectively. Under fed conditions, the mean Cₘₐₓ and AUC for the Example 1 formulation were 48.93 μg/L and 679.46 μg/L, and for the COVERA®HS were found to be 58.7 μg/L and 783.42 μg/L, respectively.

Two one-sided statistical tests were carried out using the log transformed data from the bioequivalence study at the 0.05 level of significance. The 90% confidence intervals for the Example 1 formulation and COVERA® HS were approximately within the 80% to 125% range (−20%+25%) for Cₘₐₓ and AUC. The Tₘₐₓ for the example 1 formulation was 10 hours under fed and 10.33 hours under fasting conditions, and 11 hours under fed and 11.33 under fasting for COVERA® HS, respectively. Variability was similar for the Example 1 formulation and COVERA® HS: AUC variability for the verapamil formulation of Example 1 has a %CV (coefficient of variation) of 49.14% for verapamil levels and %CV of 47.9% for COVERA® HS.

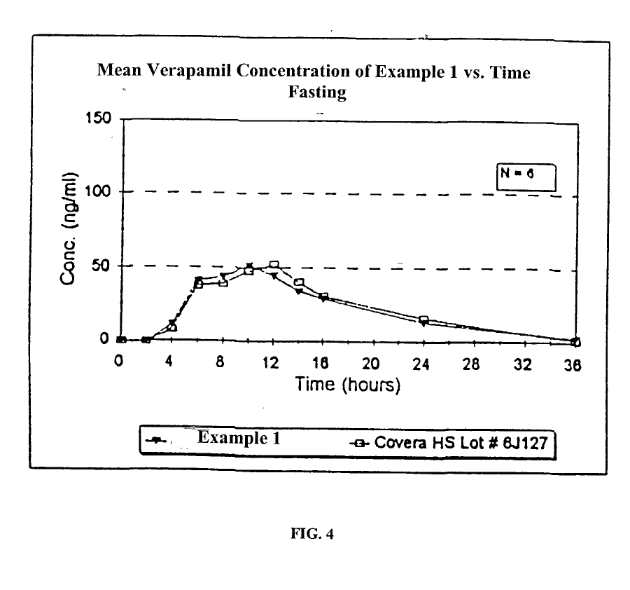

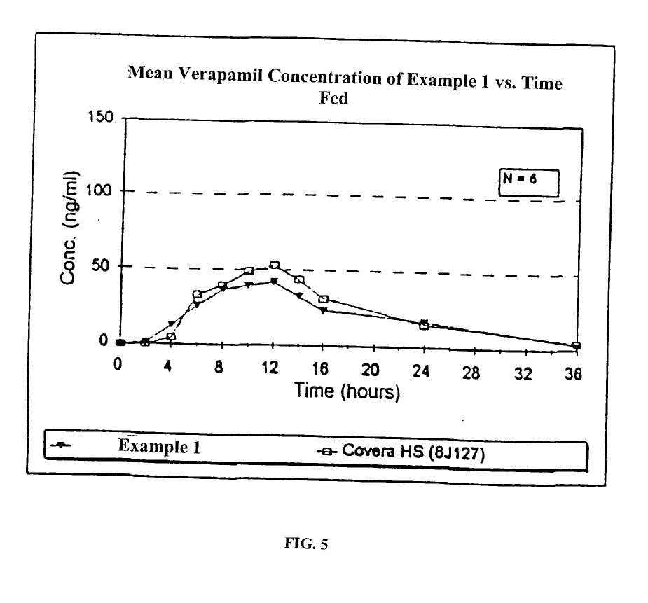

FIGS. 4 and 5 depict the in vivo verapamil plasma profile of the controlled release product prepared in Example 1 under fasting and fed conditions, respectively. Also shown in FIGS. 4 and 5 is the in vivo verapamil plasma profile of COVERA® HS.

Table 1 is a summary of the bioavailability comparison data under fasting conditions, test/reference ratio, shown in FIG. 4 wherein the COVERA HS product is the reference product in a two way crossover biostudy with n=6.

TABLE 1

| | Test Mean | Ref Mean | G-Mean Ratio |
|---|---|---|---|
| Cₘₐₓ | 64.40 | 58.80 | 0.99 |
| AUC₀₋ₜ | 727.72 | 730.28 | 0.984 |

Table 2 is a summary of the bioavailability comparison data under non-fasting conditions, test/reference ratio. Shown in FIG. 5 wherein the COVERA® HS is the reference product in a two way crossover biostudy with n=6.

TABLE 2

| | Test Mean | Ref Mean | G-Mean Ratio |
|---|---|---|---|
| Cₘₐₓ | 48.93 | 58.70 | 0.845 |
| AUC₀₋ₜ | 679.46 | 783.42 | 0.870 |

### EXAMPLE 2

It was desired to produce a formulation using the present invention together with a laser drilled release technology. The following tablets were prepared with percentages based on the weight total of the tablet.

| I ACTIVE CORE | |
|---|---|
| Verapamil HCL | 38.082% |
| Sodium Chloride | 6.671% |
| Fumaric Acid | 7.617% |
| Povidone | 4.143% |
| Polyox WSR N-80 | 19.095% |
| Polyox WSR Coagulant | 15.351% |
| Magnesium Stearate | 1.429% |
| Myvatex TL | 2.858% |
| II SEAL COATING | |
| Opadry clear | 2.946% |
| III CA COATING | |
| Cellulose Acetate 398-10 | 1.35% |
| Triacetin | 0.09% |
| Polyethylene Glycol | 0.09% |
| Sugar, confectioner's 6X-micronized | 0.27% |

IV Laser Drill

V Color Coating

Opadry White

Following the procedures of Example 1, verapamil tablets were made according to the following method:

Blend 4.042 kg of verapamil HCL, 0.808 kg of fumaric acid and 0.708 kg of sodium chloride for 15 minutes. Add 0.442 kg of povidone,USP to 10.608 kg of purified water while stirring with a homogenizer. Continue mixing for 20 minutes. The blend is then charged into a top-spray fluidized bed to wet granulate using a binding solution. After completion of the granulation step, dry for 10 minutes or until the loss on drying is less than 2%. Pass the dried granules through an 18 mesh screen. In a blender place the verapamil granules, Polyox WAR N-80, Polyox WAR-Coagulant and blend for 60 minutes. Magnesium stearate is used as a tablet lubricant. A second lubricant from the group consisting of glyceryl monostearates is added. The preferred glyceryl monostearate is Myvatex TL. Compress the blend into tablets using a Manesty Betapress with a concave tooling of 7/16″ or other suitable tableting machine. The tablets are then seal coated with Opadry Clear (YS-1-7006) to a concentration of approximately 3%. The conditions for the coating are: exhaust temperature 43±3° C., atomization pressure 50±5 psi, pan speed 4-6 rpm, spray rate 300±20g/2 guns/min.

The coating was prepared by slowing adding the cellulose acetate into acetone while homogenizing and continue mixing for 10 minutes. Add PEG 400 and triacetin into the solution, and mix for two minutes after adding each. Slowly add the confectioner's sugar into the solution. Load the verapamil seal-coated tablets into a Glatt fluidized bed coater and spray the coating suspension onto the tablets according to the following parameters: spray rate 50-150 ml/min, temperature 16-24° C., and volume 600-800 ft³/min.

Using a laser machine, a hole is drilled in the coating to further provide sustained delivery of verapamil. Typically, a 0.609 mm diameter orifice is drilled through the coating on at least one face of the tablet by a laser.

The tablets are then color coated with Opadry white.

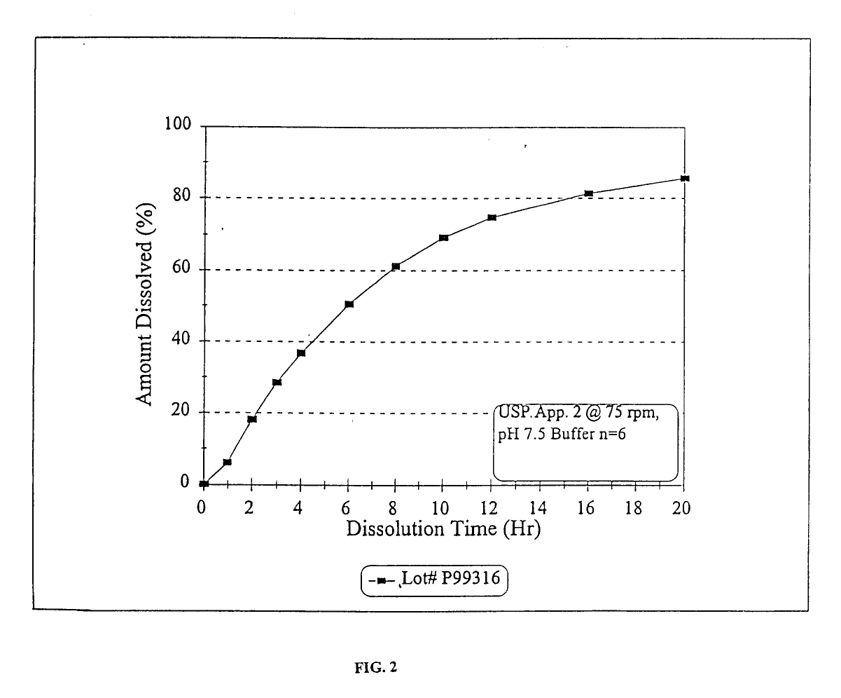

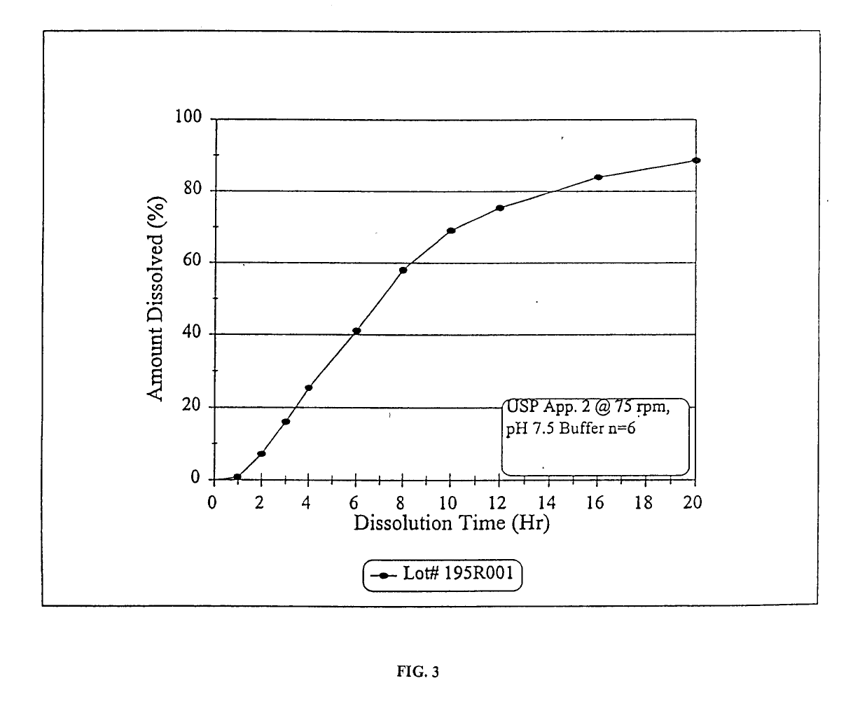

Dissolution tests on two lots of this formulation were carried out as in Example 1 using pH 7.5 buffer and the results are given in Table 3. A graphic representation of the results is indicated in FIG. 2 and FIG. 3.

TABLE 3

Verapamil HCL, 240 mg
Amount (%) Dissolved (2 different lots)

| Time (hours) | LOT A | LOT B |
|---|---|---|
| 1 | 1 | 6 |
| 2 | 7 | 18 |
| 3 | 16 | 28 |
| 4 | 25 | 37 |
| 6 | 41 | 51 |
| 8 | 58 | 61 |
| 10 | 69 | 69 |
| 12 | 76 | 75 |
| 16 | 84 | 81 |
| 20 | 89 | 86 |

While certain preferred and alternative embodiments of the invention have been set forth for purposes of disclosing the invention, modifications to the disclosed embodiments may occur to those who are skilled in the art. Accordingly, the appended claims are intended to cover all embodiments of the invention and modifications thereof which do not depart from the spirit and scope of the invention.

We claim:

1. A sustained-release pharmaceutical preparation comprising:
(a) a core comprising:
(i) a therapeutically effective dose of a calcium channel blocker;
(ii) an osmopolymer having a high and/or low molecular weight;
(iii) optionally, an osmagent;
(iv) an organic acid;
(v) optionally, a disintegrant,
(vi) optionally, a lubricant; and
(vii) optionally, at least one pharmaceutically acceptable inert ingredient,
(b) optionally, a seal coating surrounding said core, and;
(c) a water-insoluble coating comprising:
(i) a water insoluble polymer;
(ii) a plasticizer; and
(iii) a dissolution enhancing agent.

2. The sustained-release pharmaceutical preparation as defined in claim 1 wherein the calcium channel blocking agent is selected from the group consisting of verapamil, diltiazem, amlodipine, nicardipine, nimodipine, nilvadipine, flunarizine, norverapamil, nitredipine, cinnarizine, nifedipine, nisoldipine, fendiline, isradipine, feldopine, or their pharmaceutically acceptable derivatives, salts and steroisomers thereof.

3. The sustained-release pharmaceutical preparation as defined in claim 2 wherein the calcium channel blocker is verapamil or a pharmaceutically acceptable derivative thereof.

4. The pharmaceutical preparation of claim 1 wherein the disintegrant is selected from the group consisting of alginic acid, carboxymethylcellulose, croscarmelose sodium, crospovidone, povidone, sodium alginate and sodium starch glycolate.

5. The pharmaceutical preparation of claim 1 wherein the osmopolymer is a combination of high molecular weight osmopolymer and low molecular weight osmopolymer.

6. The pharmaceutical preparation of claim 5 wherein the low molecular weight osmopolymer is selected from the group consisting of polyethylene oxides, said osmopolymer having a molecular weight between 8,500 to 600,000.

7. The pharmaceutical preparation of claim 5 wherein the high molecular weight osmopolymer is selected from the group consisting of polyethylene oxides, said osmopolymer having a molecular weight over 4,000,000.

8. The pharmaceutical preparation of claim 5 wherein the low molecular weight osmopolymer is Polyox N-80.

9. The pharmaceutical preparation of claim 5 wherein the high molecular weight osmopolymer is Polyox coagulant.

10. The pharmaceutical preparation of claim 5 wherein the ratio of the amount of weight percent of the high molecular weight osmopolymer to low molecular weight osmopolymer is about 1:1.24.

11. The pharmaceutical preparation of claim 1 wherein the osmagent is selected from the group of organic and inorganic salts.

12. The pharmaceutical preparation in claim 5 wherein the tablet lubricant is selected from the group consisting of glycerol monostearates, magnesium stearate, calcium stearate, stearic acid and monoglycerides.

13. The pharmaceutical preparation of claim 1 wherein the osmagent is sodium chloride.

14. The pharmaceutical preparation of claim 1 wherein the organic acid is selected from the group consisting of adipic acid, ascorbic acid, citric acid, fumaric acid, malic acid, succinic acid, tartaric acid or a mixture thereof.

15. The pharmaceutical preparation of claim 1 wherein the organic acid is fumaric acid.

16. The pharmaceutical preparation in claim 1 wherein the polymer in the water insoluble coating is selected from the group consisting of cellulose polymers.

17. The pharmaceutical preparation of claim 16 wherein the cellulose polymer selected is cellulose acetate.

18. The pharmaceutical preparation in claim 17 wherein the amount of cellulose acetate in the semipermeable coating is greater than about 60 wt %.

19. The pharmaceutical preparation in claim 1 wherein the plasticizers comprise 10-20% of the water-insoluble coating.

20. The pharmaceutical preparation in claim 1 wherein the plasticizer is a combination of plasticizers wherein one is an organic ester.

21. The pharmaceutical preparation according to claim 20 wherein the combination of plasticizers is 1:1.

22. The pharmaceutical preparation according to claim 20 wherein the organic ester of plasticizer is triacetin.

23. The pharmaceutical preparation according to claim 1 where the dissolution enhancing agent is a polyethylene glycol having a molecular weight range of about 100 to 7500.

24. The pharmaceutical preparation according to claim 1 wherein the plasticizer is PEG 400.

25. The sustained-release pharmaceutical preparation as defined in claim 1 wherein the core comprises:
30-50% of the calcium channel blocker;
2-10% of the organic acid;
10-35% of the low molecular weight osmopolymer;
10-20% of the high molecular weight osmopolymer;
2-10% of the disintegrant;
2-10% of the osmagent;
0.1-2% of the lubricant.

26. The sustained-release pharmaceutical preparation as defined in claim 25 wherein the core comprises:
35-45% of the calcium channel blocker;
4-8% of the organic acid;
15-25% of the low molecular weight osmopolymer;
13-18% of the high molecular weight osmopolymer;
3-6% of the disintegrant;
3-8% of the water soluble osmotic agent;
0.5-1.5% of the lubricant.

27. The pharmaceutical preparation according to claim 26 with at least one exit means connecting the core with the exterior of the sustained release dosage formulation.

28. The pharmaceutical preparation according to claim 1 wherein the semipermeable coating consists essentially of:
70-80% cellulose acetate;
3-10% triacetin;
3-10% PEG 400; and
10-20% sugar.

29. The sustained-release pharmaceutical formulation as defined in claim 1 that exhibits the following dissolution profile when tested in a USP type 2 apparatus at 75 rpm in simulated intestinal fluid (pH 6.8 buffer) at 37° C.:
after 2 hours 0-25% of the drug is released;
after 4 hours 10-50% of the drug is released;
after 8 hours 30-80% of the drug is released;
not less than 50% of the drug is released after 12 hours;
not less than 60% of the drug is released after 16 hours.

30. The sustained-release pharmaceutical formulation as defined in claim 1 that exhibits the following dissolution profile when tested in a USP type 2 apparatus at 75 rpm in simulated intestinal fluid (pH 6.8 buffer) at 37° C.:
after 2 hours 5-20% of the drug is released;
after 4 hours 15-40% of the drug is released;
after 8 hours 40-70% of the drug is released;
not less than 55% of the drug is released after 12 hours;
not less than 65% of the drug is released after 16 hours.

31. The sustained-release pharmaceutical formulation as defined in claim 1 that exhibits the following dissolution profile when tested in a USP type 2 apparatus at 75 rpm in simulated gastric fluid (pH 1.2) at 37° C.:
after 2 hours 0-25% of the drug is released;
after 4 hours 10-40% of the drug is released;
after 8 hours 40-75% of the drug is released;
not less than 50% of the drug is released after 12 hours;
not less than 65% of the drug is released after 16 hours.

32. The sustained-release pharmaceutical formulation as defined in claim 1 that exhibits the following dissolution profile when tested in a USP type 2 apparatus at 75 rpm in simulated gastric fluid (pH 1.2) at 37° C.:
after 2 hours 5-20% of the drug is released;
after 4 hours 15-35% of the drug is released;
after 8 hours 50-65% of the drug is released;
not less than 60% of the drug is released after 12 hours;
not less than 65% of the drug is released after 16 hours.

33. The sustained-release pharmaceutical preparation wherein the core consists essentially of:
30-50% of the calcium channel blocker;
2-10% of the organic acid;
10-35% of the low molecular weight osmopolymer;
10-20% of the high molecular weight osmopolymer;
2-10% of the disintegrant;
2-10% of the osmagent;
0.1-2% of the lubricant; and
the semipermeable coating consists essentially of:
70-80% water-insoluble polymer;
3-20% plasticizer; and
10-20% dissolution enhancing agent.

34. The sustained-release pharmaceutical formulation as defined in claim 33 that exhibits the following dissolution profile when tested in a USP type 2 apparatus at 75 rpm in simulated intestinal fluid (pH 6.8 buffer) at 37° C.:
after 2 hours 0-25% of the drug is released;
after 4 hours 10-50% of the drug is released;
after 8 hours 30-80% of the drug is released;
not less than 50% of the drug is released after 12 hours;
not less than 60% of the drug is released after 16 hours.

35. The sustained-release pharmaceutical formulation as defined in claim 33 that exhibits the following dissolution profile when tested in a USP type 2 apparatus at 75 rpm in simulated intestinal fluid (pH 6.8 buffer) at 37° C.:
after 2 hours 5-20% of the drug is released;
after 4 hours 15-40% of the drug is released;
after 8 hours 40-70% of the drug is released;
not less than 55% of the drug is released after 12 hours;
not less than 65% of the drug is released after 16 hours.

36. The sustained-release pharmaceutical formulation as defined in claim 33 that exhibits the following dissolution profile when tested in a USP type 2 apparatus at 75 rpm in simulated gastric fluid (pH 1.2) at 37° C.:
after 2 hours 0-25% of the drug is released;
after 4 hours 10-40% of the drug is released;
after 8 hours 40-75% of the drug is released;
not less than 50% of the drug is released after 12 hours;
not less than 65% of the drug is released after 16 hours.

37. The sustained-release pharmaceutical formulation as defined in claim 33 that exhibits the following dissolution profile when tested in a USP type 2 apparatus at 75 rpm in simulated gastric fluid (pH 1.2) at 37° C.:
after 2 hours 5-20% of the drug is released;
after 4 hours 15-35% of the drug is released;
after 8 hours 50-65% of the drug is released;
not less than 55% of the drug is released after 12 hours;
not less than 65% of the drug is released after 16 hours.
